(19) United States
(12) Patent Application Publication
(10) Pub. No.: US 2026/0134998 A1
(43) Pub. Date: May 14, 2026

(54) CLINICAL WORKFLOW ESCALATION AND ACKNOWLEDGMENT GOVERNANCE SYSTEM FOR AI-ASSISTED MEDICAL DECISION SUPPORT

(71) Applicant: George William Bickerstaff, III, Greenwich, CT (US)

(72) Inventor: George William Bickerstaff, III, Greenwich, CT (US)

(21) Appl. No.: 19/445,561

(22) Filed: Jan. 11, 2026

(51) Int. Cl. G16H 50/20

(52) U.S. Cl. CPC G16H 50/20

(57) ABSTRACT

A clinical workflow escalation and acknowledgment governance system converts AI-assisted clinical outputs into governed workflow items. The system enforces execution-time routing, tiered escalation, and acknowledgment thresholds and stores immutable audit records. The invention improves safety and accountability without autonomously diagnosing conditions or prescribing treatment actions.

### TECHNICAL FIELD

The present invention relates to computer-implemented healthcare governance systems and, more particularly, to execution-time workflow escalation, acknowledgment enforcement, and accountability control for artificial intelligence-assisted medical decision support systems operating within regulated clinical environments.

### BACKGROUND

Artificial intelligence systems are increasingly used in healthcare settings to generate alerts, risk indicators, prioritizations, and clinical support outputs derived from patient data and operational context.

While such systems may improve awareness and efficiency, they also introduce operational and patient-safety risk when AI-generated outputs are delivered without structured workflow governance, escalation logic, or acknowledgment enforcement.

Existing alerting and notification systems frequently rely on passive delivery mechanisms that do not guarantee timely review, documented acknowledgment, or escalation to appropriate clinical roles.

This deficiency can result in missed alerts, delayed intervention, alert fatigue, and ambiguity regarding responsibility for responding to AI-assisted clinical outputs.

Accordingly, there exists a need for a technical system that converts AI-generated outputs into governed workflow items, enforces escalation and acknowledgment rules at execution time, and produces immutable audit records demonstrating how and when clinical workflows were handled, without autonomously diagnosing medical conditions or prescribing treatment actions.

### SUMMARY

The disclosed invention provides a computer-implemented clinical workflow escalation and acknowledgment governance system that receives outputs from one or more artificial intelligence clinical decision support systems and transforms the outputs into structured workflow items governed by execution-time escalation logic.

The system enforces acknowledgment thresholds, role-based routing, tiered escalation, fail-safe escalation, deferral handling, and resolution requirements and records clinician interactions in immutable, time-stamped accountability records.

The system operates strictly as a governance and workflow control layer and does not autonomously diagnose medical conditions, recommend treatment, or replace clinician judgment.

### DEFINITIONS (ALPHABETICAL ORDER)

Acknowledgment Threshold means a predefined execution-time condition, including elapsed time or event occurrence, that triggers escalation of a workflow item.
AI Clinical Decision Support System means a computational system that generates clinical support outputs without autonomously diagnosing or treating patients.
Audit Record means an immutable, time-stamped record of workflow state changes, escalation events, and clinician interactions.
Clinician interaction means an explicit action performed by an authorized healthcare professional in response to a workflow item, including acknowledgment, deferral, override, re-routing, or resolution.
Escalation Rule means a configurable execution-time condition governing routing of a workflow item to one or more clinical roles.
Execution-Time Governance means enforcement of workflow control logic prior to or during clinical operations such that workflow progression, routing, or visibility is conditioned on system rules and recorded events.
Workflow Item means a system-managed representation of an AI-generated clinical support output.
Workflow Orchestration Engine means a software component that assigns, updates, and governs workflow states and routes workflow items.
Workflow Priority means a classification influencing routing order and escalation timing.
Workflow Resolution means completion of handling for a workflow item such that no further escalation is required.
Workflow State means a defined execution status governing how a workflow item is handled.

### BRIEF DESCRIPTION OF THE DRAWINGS

FIG. 1 illustrates a clinical workflow escalation governance system architecture.

FIG. 2 illustrates AI output ingestion and workflow state generation.

FIG. 3 illustrates escalation, routing, and acknowledgment enforcement.

FIG. 4 illustrates clinician interaction, deferral, override, and workflow resolution.

FIG. 5 illustrates audit logging, reporting, and compliance outputs.

### DETAILED DESCRIPTION

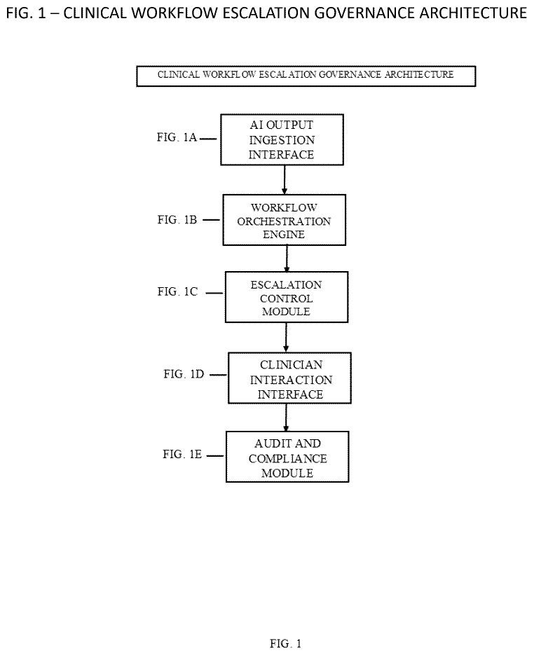

### FIG. 1—Clinical Workflow Escalation Governance Architecture

FIG. 1 illustrates a clinical workflow escalation governance system comprising AI integration interfaces, a workflow orchestration engine, escalation control logic, clinician interaction interfaces, and audit and compliance components. The architecture separates AI output generation from workflow governance to ensure controlled execution-time handling. The system may operate within on-premises infrastructure, distributed computing environments, or secure cloud services.

### FIG. 1A—AI Output Ingestion Interface

FIG. 1A illustrates an interface configured to receive AI-assisted clinical outputs from one or more AI clinical decision support systems. The interface normalizes outputs into standardized workflow items containing required metadata. Normalization enables consistent workflow governance independent of model source.

### FIG. 1B—Workflow Orchestration Engine

FIG. 1B illustrates a workflow orchestration engine configured to assign workflow states and manage state transitions. The engine enforces execution-time governance by conditioning routing and escalation on recorded events. The engine does not perform clinical diagnosis or treatment selection.

### FIG. 1C—Escalation Control Module

FIG. 1C illustrates an escalation control module that evaluates workflow items against acknowledgment thresholds and escalation rules. The module triggers tiered escalation when thresholds are unmet. Escalation actions increase visibility without mandating clinical action.

### FIG. 1D—Clinician Interaction Interface

FIG. 1D illustrates an interface enabling clinicians to acknowledge, defer, override workflow handling, re-route, or resolve workflow items. Interactions are structured and recorded. AI outputs remain unchanged.

### FIG. 1E—Audit and Compliance Module

FIG. 1E illustrates an audit module that records workflow events as immutable records. Records support compliance and review. Stored data is tamper-resistant.

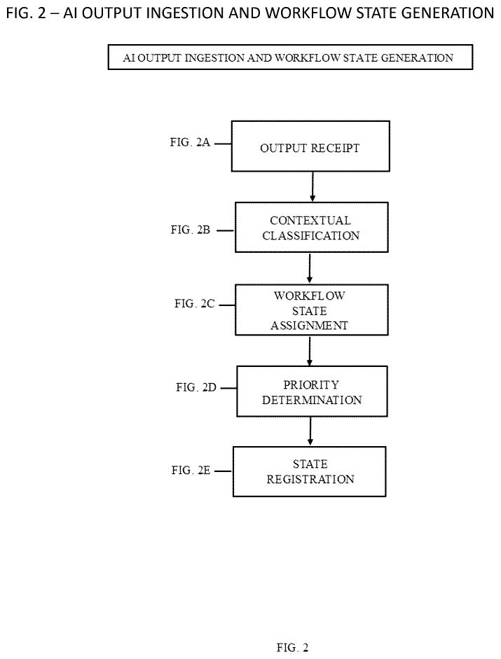

### FIG. 2—AI Output Ingestion and Workflow State Generation

FIG. 2 illustrates conversion of AI outputs into governed workflow items. Workflow states are assigned using institutional configuration and context. Governance controls entry into clinical operations.

### FIG. 2A—Output Receipt

FIG. 2A illustrates receipt of an AI-generated output. Metadata may accompany the output. Receipt does not trigger treatment.

### FIG. 2B—Contextual Classification

FIG. 2B illustrates contextual classification of outputs using non-diagnostic context. Classification informs routing logic. Classification does not alter AI inference.

### FIG. 2C—Workflow State Assignment

FIG. 2C illustrates assignment of predefined workflow states. States govern escalation behavior. State assignment is deterministic.

### FIG. 2D—Priority Determination

FIG. 2D illustrates priority determination influencing escalation timing. Priority is configurable. Priority does not imply treatment urgency.

### FIG. 2E—State Registration

FIG. 2E illustrates registration of workflow state for tracking and auditability. The state persists until updated.

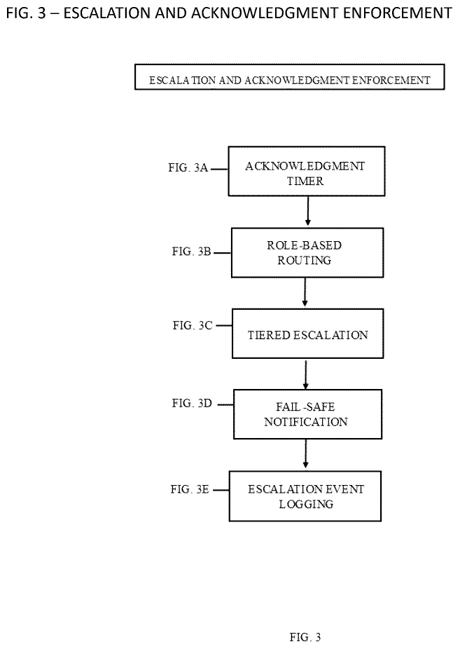

### FIG. 3—Escalation and Acknowledgment Enforcement

FIG. 3 illustrates execution-time escalation and acknowledgment enforcement. Workflow items escalate if thresholds are unmet. Escalation preserves clinician discretion.

FIG. 3A—Acknowledgment Timer

FIG. 3A illustrates initiation of an acknowledgment timer. Timers measure elapsed time. Thresholds are configurable.

### FIG. 3B—Role-Based Routing

FIG. 3B illustrates routing to designated clinical roles. Routing respects institutional configuration. Routing does not assign clinical responsibility.

### FIG. 3C—Tiered Escalation

FIG. 3C illustrates tiered escalation when acknowledgment thresholds are exceeded. Additional roles are notified. Escalation continues until acknowledged.

### FIG. 3D—Fail-Safe Notification

FIG. 3D illustrates fail-safe escalation ensuring visibility of high-priority items. Fail-safe logic is auditable.

### FIG. 3E—Escalation Event Logging

FIG. 3E illustrates logging of escalation events. Logs capture timing and recipients. Logs are immutable.

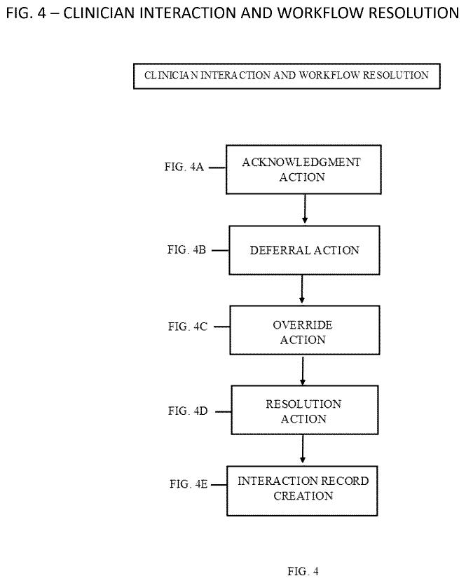

### FIG. 4—Clinician Interaction and Workflow Resolution

FIG. 4 illustrates clinician handling of workflow items. Actions are explicit and documented. Clinical authority remains with humans.

### FIG. 4A—Acknowledgment Action

FIG. 4A illustrates clinician acknowledgment. Acknowledgment confirms review. Timing is recorded.

### FIG. 4B—Deferral Action

FIG. 4B illustrates deferral with optional conditions. Deferrals are tracked. Escalation may resume upon expiration.

### FIG. 4C—Override Action

FIG. 4C illustrates override of workflow handling. Overrides document judgment. AI outputs remain unchanged.

### FIG. 4D—Resolution Action

FIG. 4D illustrates resolution of workflow items. Resolution ends escalation. Reasons may be recorded.

### FIG. 4E—Interaction Record Creation

FIG. 4E illustrates creation of immutable interaction records. Records bind identity, action, and timing. Records support accountability.

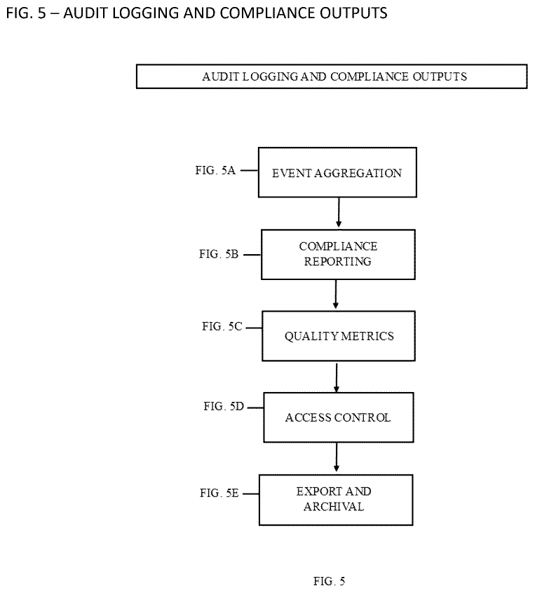

### FIG. 5—Audit Logging and Compliance Outputs

FIG. 5 illustrates audit and compliance outputs derived from workflow handling. Outputs support governance. Outputs do not interfere with care.

### FIG. 5A—Event Aggregation

FIG. 5A illustrates aggregation of workflow events. Aggregation supports analysis. Individual records remain intact.

### FIG. 5B—Compliance Reporting

FIG. 5B illustrates generation of compliance reports. Reports summarize escalation and response. Reports are regulator-ready.

### FIG. 5C—Quality Metrics

FIG. 5C illustrates derivation of quality metrics. Metrics include acknowledgment latency. Metrics inform improvement.

### FIG. 5D—Access Control

FIG. 5D illustrates role-based access control. Access is logged. Unauthorized access is prevented.

### FIG. 5E—Export and Archival

FIG. 5E illustrates export and archival of records. Records are retained per policy. Export supports audits.

### EXAMPLE

In one example, an AI system generates a stroke risk alert. The system assigns a review-required workflow state and initiates an acknowledgment timer. The alert escalates to additional clinicians until acknowledged.

A clinician acknowledges and defers the item pending imaging. Upon deferral expiration, escalation resumes and the item is re-routed. The item is later resolved and all events are logged.

Audit reports demonstrate escalation timing and response. The system does not diagnose or prescribe treatment.

1. A computer-implemented clinical workflow escalation governance system, comprising:
one or more processors; and
non-transitory memory storing instructions that, when executed by the one or more processors, cause the system to:
(a) receive, from an artificial intelligence clinical decision support system, an AI-assisted clinical output;
(b) generate a workflow item associated with a workflow state selected from a plurality of predefined workflow states;
(c) assign a workflow priority to the workflow item;
(d) initiate an acknowledgment timer based on the workflow priority;
(e) determine that an acknowledgment threshold is unmet;
(f) execute tiered escalation by routing the workflow item to at least one additional clinical role according to an escalation rule; and
(g) store immutable, time-stamped audit records of workflow state changes, escalation events, and clinician interactions,
wherein the system operates as a governance layer and does not autonomously diagnose a medical condition or prescribe treatment.

2. A computer-implemented method comprising:
(a) receiving an AI-assisted clinical output;
(b) assigning a workflow state and workflow priority;
(c) initiating an acknowledgment timer;
(d) escalating the workflow item upon expiration of an acknowledgment threshold;
(e) receiving a clinician interaction; and
(f) writing immutable audit records of workflow and escalation events, wherein the method enforces execution-time workflow governance without autonomously diagnosing or prescribing treatment.

3. A non-transitory computer-readable medium storing instructions
that cause one or more processors to perform the method of claim 2.

4. The system of claim 1, wherein escalation comprises sequential and parallel escalation tiers.

5. The system of claim 1, wherein fail-safe escalation is executed when routing targets are unavailable.

6. The system of claim 1, wherein deferral pauses escalation until a condition expires.

7. The system of claim 1, wherein override re-routes or reprioritizes workflow items without altering AI output.

8. The method of claim 2, wherein escalation rules depend on care setting or staffing schedules.

9. The method of claim 2, further comprising generating compliance reports.

10. The system of claim 1, wherein access to audit records is role-restricted and logged.
